(19) United States
(12) Patent Application Publication
(10) Pub. No.: US 2007/0270876 A1
(43) Pub. Date: Nov. 22, 2007

(54) Vertebra bone cement introduction system

(76) Inventors: Yi-Chen Kuo, Taipei (TW); Shu-Hui Yang, Taipei (TW)

Correspondence Address: BIRCH STEWART KOLASCH & BIRCH, PO BOX 747, FALLS CHURCH, VA, 22040-0747, US

(21) Appl. No.: 11/399,534

(22) Filed: Apr. 7, 2006

(51) Int. Cl. A61F 2/00

(52) U.S. Cl. 606/92

(57) ABSTRACT

A vertebra bone cement introduction system includes a main frame comprised of lateral handle and vertical insertion tube; a pouring frame comprised of lateral handle and vertical extension tube; an external injection device to inject cement into extension tube; a taper being meshed to extension tube; a polygonal hole disposed to taper and a recession in the mid of taper connected to a silicon gel bag containing cement; a locking rod penetrating the extension tube; and the lateral rod of the pouring frame being turned in reverse to disengage taper from extension tube while leaving taper and bag in a hole drilled on the bone.

### FIELD OF THE INVENTION

The present invention is related to a vertebra bone cement introduction system, and more particularly, to one that allows easy operation and effective prevention of the bone cement from dispersion.

### BACKGROUND OF THE INVENTION

Insufficient assimilation of calcium in diet results in that more and more people today suffer osteoporosis. Whereas one gets older, the bone fails to promote calcium containment, bone cement is introduced into vital parts of the bone (e.g., the vertebra to improve bone strength for preventing the damages osteoporosis could do to our body.

The conventional way of introducing the bone cement into the vertebra is done by first having drilled a hole in proper size on the surface of the bone (vertebra), a metal catheter is directly inserted through the hole, and an external barrel is used to inject with a proper pressure a liquid cement at high temperature into the hole via the metal catheter. Once cured, the bone cement becomes a comparatively harden block to fill up the hole, usually located at where osteoporosis is diagnosed. However the pouring method of bone cement of the prior art is found with the following flaws:
1. Whereas the bone cement when poured is in the form of liquid at high temperature, it tends to have improper dispersion to affect the effects of improved bone strength.
2. Improper dispersion of the bone cement in liquid state at high temperature when poured into the bone will damage the peripheral nerves for the patient to suffer hazard of improper treatment.
3. Whereas the hole drilled on the surface of the bone relates to an open space connecting through the interior of the bone where osteoporosis is diagnosed, the operator has to be very careful with the amount of bone cement to be poured into the hole since any excessive amount may lead to leakage and contamination. Meanwhile, the operation precision and convenience are compromised by the outward push generated by the metal catheter under the pressure of the pouring of the bone cement.

### SUMMARY OF THE INVENTION

The primary purpose of the present invention is to provide a bone cement introduction system that prevents improper dispersion of the cement in the bone to effectively upgrade results of improved bone strength and prevents possible damage to the peripheral nerves due to improper dispersion of the cement at high temperature in the bone.

Another purpose of the present invention is to provide a bone cement introduction system that allows easy and precision operation to effectively upgrade its quality of medical treatment.

To achieve those purposes, the present invention is essentially comprised of a main frame, a pouring frame, and a locking tube. The main frame includes a lateral handle and a insertion tube vertically inserted through the mid of the handle with the lower end of the insertion tube to penetrate into a hole drilled on the surface of a bone. The pouring frame includes a lateral handle and an extension tube vertically inserted through the lateral handle. The lower end of the extension tube is internally threaded and inserted into the tube of the main frame and further into the hole. The lateral handle of the pouring frame connecting through the opening of the extension tube is connected with an external injection device to pour with proper pressure the bone cement in liquid and high temperature status into the extension tube, and a taper having its outer circumference externally threaded to mesh to the internal thread at the lower end of the extension tube of the pouring frame to extend into the hole on the bone. A polygonal is provided on one side at the end of the taper meshed to the extension tube and a recession is disposed to the middle section of the outer circumference of the taper to be adapted to a silicon gel bag containing the bone cement. The locking rod penetrates through the extension tube of the pouring frame and has at its lower end disposed with a polygonal protrusion to be mated into the polygonal hole of the taper for the lateral handle of the pouring frame to turn in reverse so to clear the outer thread of the taper off the inner thread.

### BRIEF DESCRIPTION OF THE DRAWINGS

FIG. 1 is an exploded view showing certain elements of the present invention.

FIG. 2 is an exploded view showing other elements of the present invention.

FIG. 3 is a first schematic view showing the operation of the present invention.

FIG. 4 is a second schematic view showing the operation of the present invention.

FIG. 5 is a third schematic view showing the operation of the present invention.

FIG. 6 is a fourth schematic view showing the operation of the present invention.

FIG. 7 is a fifth schematic view showing the operation of the present invention.

FIG. 8 is a sixth schematic view showing the operation of the present invention.

FIG. 9 is a seventh schematic view showing the operation of the present invention.

FIG. 10 is a schematic view showing partial construction of the present invention.

### DETAILED DESCRIPTION OF THE PREFERRED EMBODIMENTS

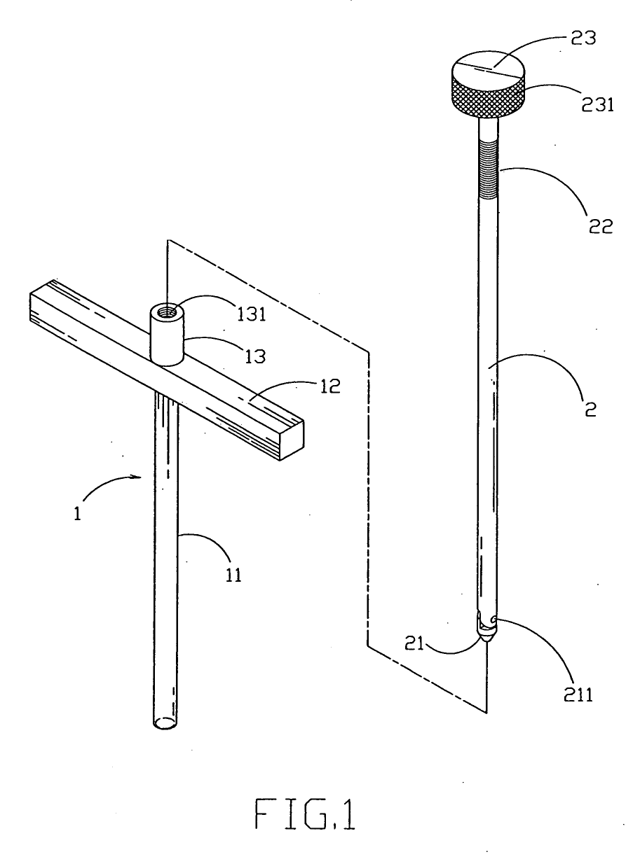

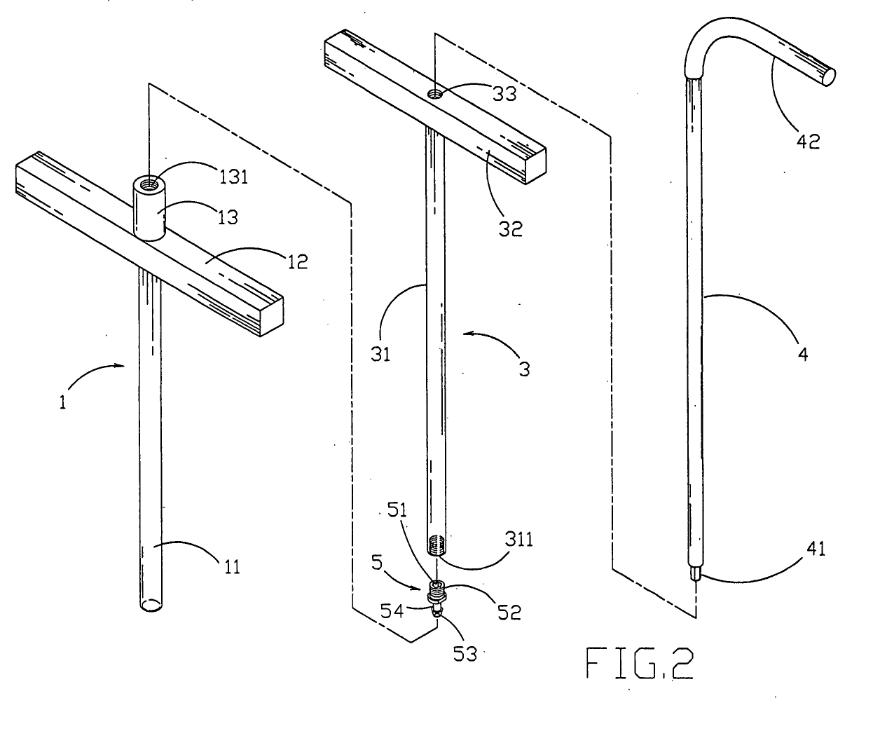

Referring to FIGS. 1 and 2, the present invention is essentially comprised of a main frame 1, an expansion rod 2, a pouring frame 3, a locking rod 4, a taper 5, and a silicone gel bag 7. The main frame 1 includes a lateral handle 12 and an insertion tube 11 penetrating through a the mid section of the lateral handle 12, an upper section 13 of the insertion tube 11 above the lateral handle 12 is provided with an internally threaded section 131 to connect through the insertion tube 11. One end of the expansion rod 2 is disposed with a knob 23 and a pivoting head 21 is pivoted to another end of the expansion rod 2 by means of a pivot 211. A certain section of the expansion rod 2 at where closer to the end provided with the knob 23 relates to an externally threaded section 22. An extension tube 31 vertically penetrates through the mid of a lateral handle 32 of the pouring frame 3. An internally threaded section 33 is provided to an opening where the lateral handle 32 connects through the extension tube 31 and an internally threaded section 311 is disposed on the other end of the extension tube 31. An externally threaded section 52 is provided to the outer circumference of one end of the taper 5 to mesh to the internally threaded section 311 at the lower end of the extension tube 31 of the pouring frame 3.

A polygonal hole 51 is disposed to the end of the taper 5 at where it is coupled to the extension tube 31; and another end of the taper 5 relates to a conic head 53. A recession 54 is disposed to the terminal of the expansion portion of the conic head 53 to receive the adaptation of the silicone gel bag 7. One end of the locking rod 4 is provided with a polygonal protrusion 41 and a curved handle 42 is provided to another end at where exposed out of the extension tube 31 of the pouring frame 3.

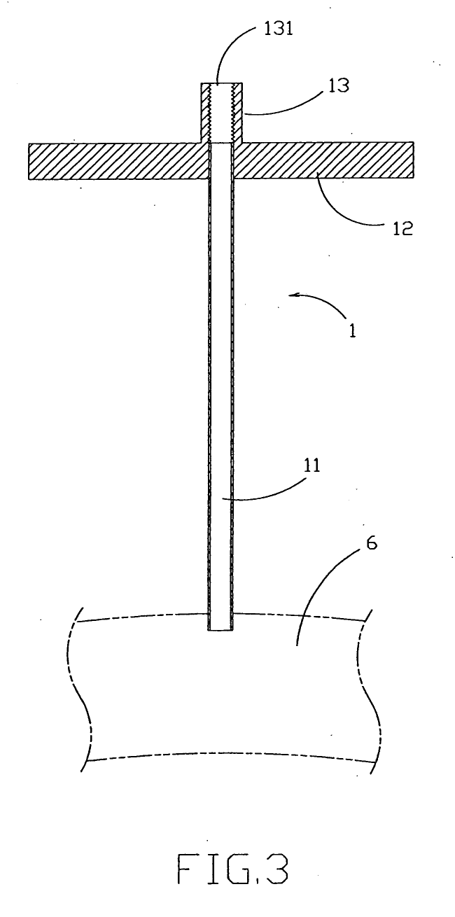

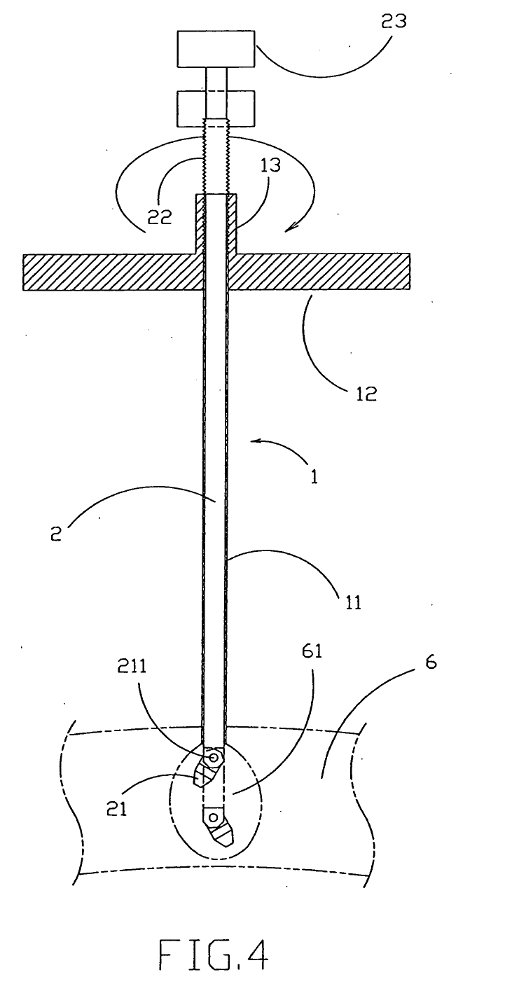

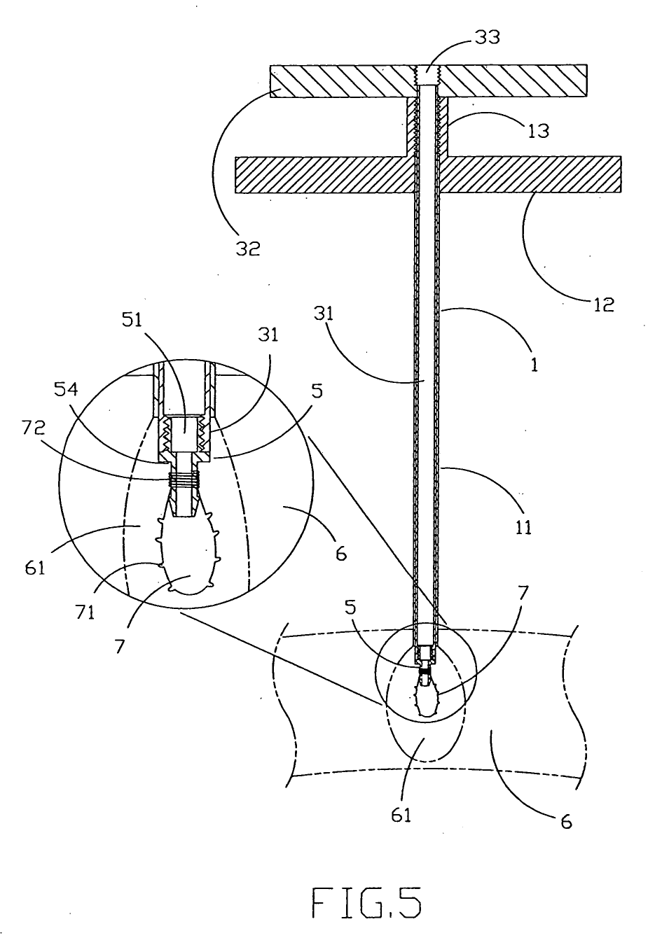

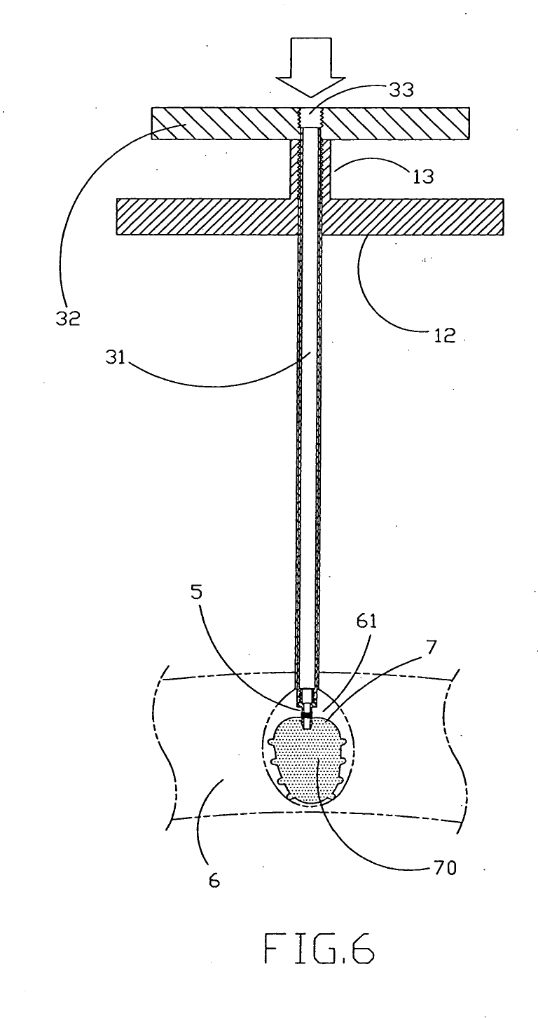

FIGS. 3 through 9 shows the operation of the present invention. Firstly, the out end of the insertion tube 11 of the main frame 1 is inserted into a shallow hole predrilled on the surface of a bone 6 as illustrated in FIG. 3., and the expansion rod 2 penetrates through the insertion rube 11 of the main frame 1 for the pivoting head 21 to enter into the shallow hole on the surface of the bone 6 before having the externally threaded section 22 of the expansion rod 2 meshed to the internally threaded section 131 of the main frame 1. While the expansion rod 2 is turned during the meshing, the turning pivoting head 21 creates deflection for the expansion rod 2 slowly pushes the pivoting head 21 when both internally and externally sections are meshed. Accordingly, the pivoting head 21 expands the hole in the bone 6 to define an accommodation space 61 as illustrated in FIG. 4. The expansion rod 2 is then withdrawn from the insertion tube 11 of the main frame 1. The taper 5 is coupled to the pouring frame 3 at the lower end of the extension tube 31. The silicon gel bag 7 provided with multiple flanges 71 is fastened to the circumference of the recession 54 of the taper 5 by means of multiple loops of a thread 72, so that the pouring frame 3 has its extension tube 31 to penetrate through the insertion tube 11 of the main frame 1 for the taper 5 and the silicon gel bag 7 to extend into the accommodation space 61 of the bone 6 as illustrated in FIG. 5. An external injection device (e.g., a barrel) to pour a bone cement 70 through the extension tube 31 of the pouring frame 3, the taper 5, and into the silicone gel bag 7 for the silicon gel bag 7 to gradually expand due to the pressure applied by the bone cement 70 as illustrated in FIG. 6.

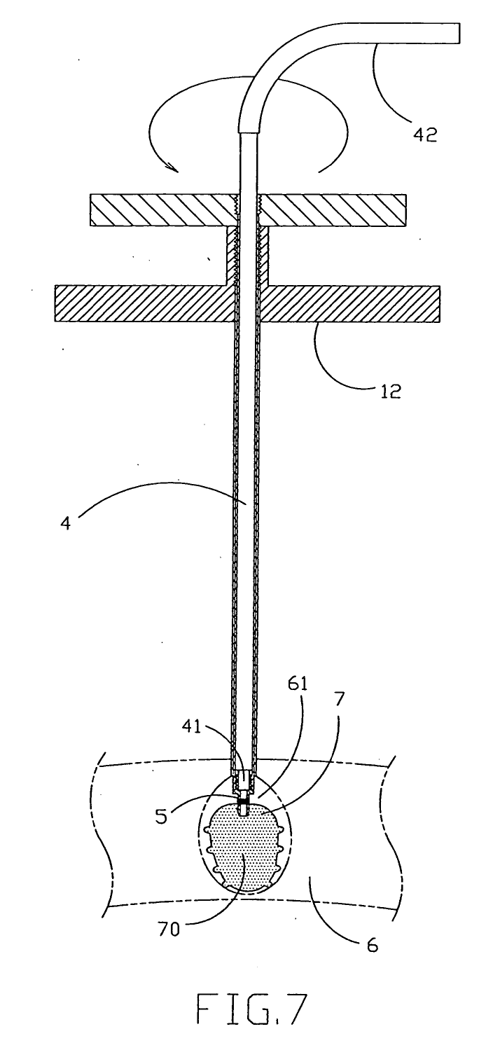

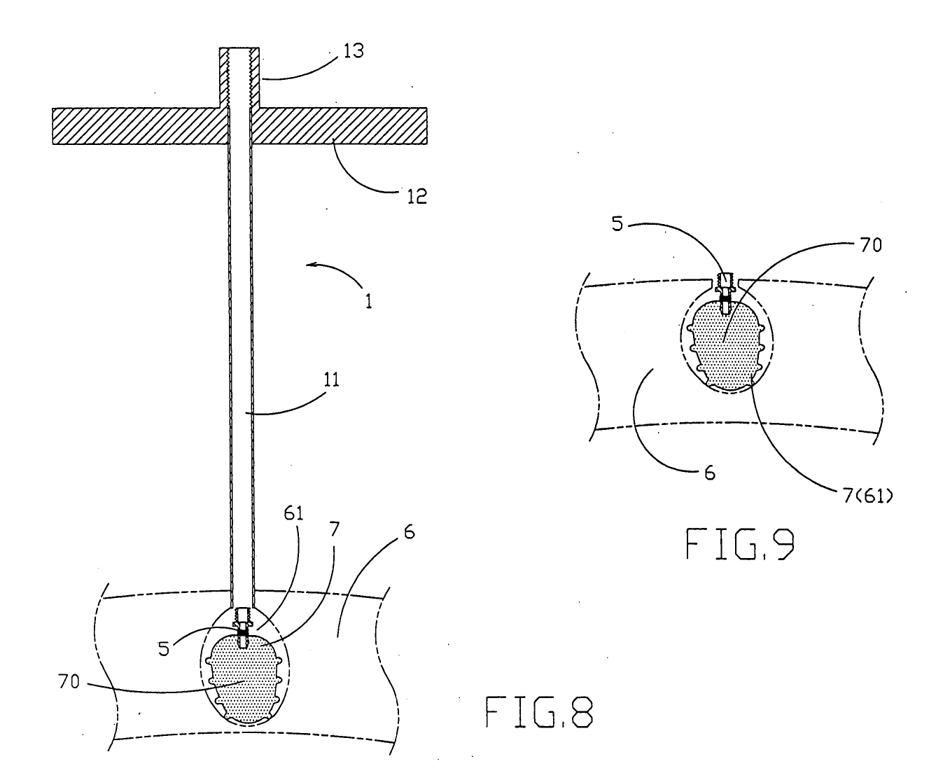

Upon the completion of the filling of the bone cement 70 at a given amount , the end of the locking rod 4 provided with the polygonal protrusion 41 penetrates through the extension tube 31 of the pouring frame 3 to force any remaining bone cement 70 in the extension tube 31 into the silicon gel bag 7 and to insert and secure the polygonal protrusion 41 in the polygonal hole 51 of the taper 5 for the lateral handle 32 of the pouring frame 3 to turn in reverse to disengage the internally threaded section 311 at the lower end of the extension tube 31 from the externally threaded section 52 of the taper 5 as illustrated in FIG. 7. The polygonal protrusion 41 of the locking rod 4 retracts from the insertion tube 11 of the main frame 1 together with the extension tube 31 of the pouring frame 3 as illustrated in FIG. 8. Finally, while the insertion tube 11 of the main frame 1 is externally withdrawn, the silicon gel bag 7 expands properly due to the pressure applied by the bone cement 70 so to fill up the accommodation space 61 of the bone 6 as illustrated in FIG. 9. Meanwhile, those multiple flanges 71 create comparatively greater friction against the inner wall of the accommodation space 61 to firmly secure the bag in position.

While the handle 42 of the locking rod 4 indicates an L shaped curvature for extension as illustrated, the handle 42 may be directly connected to the locking rod 4 in a T shape. The externally threaded end of the external injection device (the barrel) allows firm incorporation with the internally threaded section 33 of the pouring frame 3 to prevent leak of the bone cement 70. The outer circumference to the knob 23 of the expansion rod 2 is embossed 231 to allow more reliable friction for the operator to grip on.

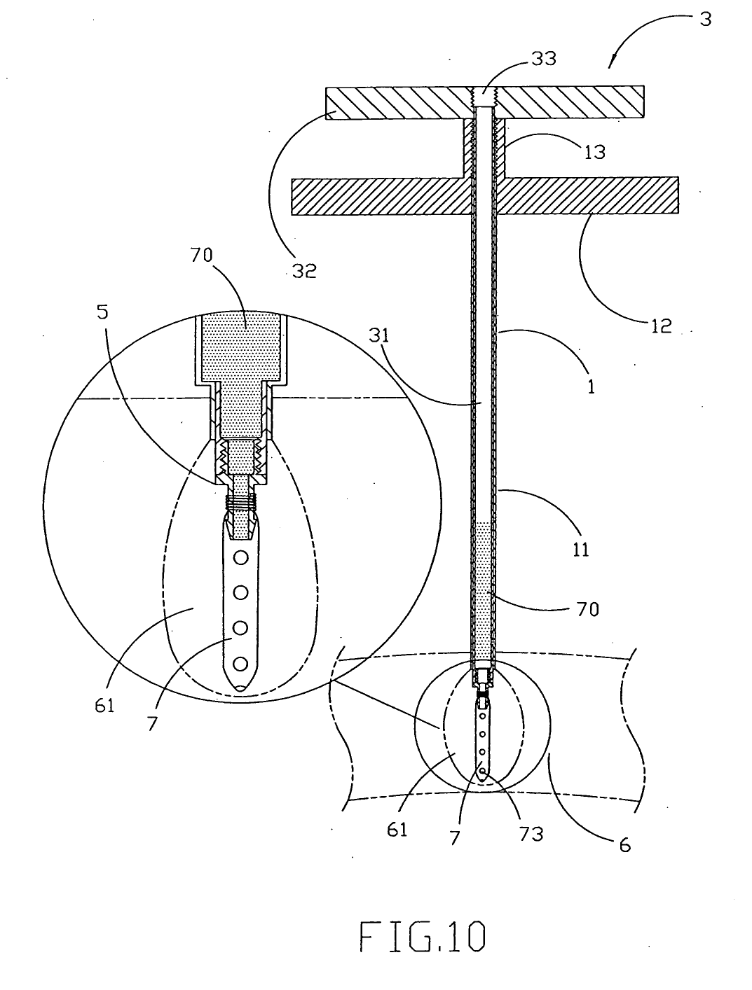

Now referring to FIG. 10, the silicon gel bag7 in another preferred embodiment is made into a strip form provided thereon one or multiple through hole 73. When the bone cement 70 is poured into the silicon gel bag 7 through the extension tube 31 of the pouring frame 3 and further through the taper 5 to cause the silicon gel bag 7 to gradually expand, the bone cement 70 is forced to slowly flow into the accommodation space 61 of the bone 6 via the through hole 73 when subject to the squeezing by the expanding silicon gel bag 7. Accordingly, the bone cement 70 is prevented from improper dispersion in the bone 6 to effectively upgrade the strength of the bone 6 while avoiding the damage to the peripheral nerves due to improper dispersion of the bone cement 70 at high temperature.

The prevent invention provides an improved structure of a vertebra bone cement introduction system, and the application for a utility patent is duly filed accordingly. However, it is to be noted that the preferred embodiments disclosed in the specification and the accompanying drawings are not limiting the present invention; and that any construction, installation, or characteristics that is same or similar to that of the present invention should fall within the scope of the purposes and claims of the present invention.

1. A vertebra bone cement introduction system includes a main frame, a pouring frame, a taper, and a locking rod; the main frame including a lateral handle and a insertion tube, the insertion tube being vertically inserted through the mid of the lateral handle, and the lower end of the insertion tube penetrating into a hole predrilled on the surface of a bone; the pouring frame including a lateral handle having at its mid section vertically inserted with an extension tube; the lower end of the extension tube being internally threaded; the extension tube penetrating the insertion tube of the main frame; the internally threaded lower end of the extension tube extending into the hole of the bone, the opening of the lateral handle connecting through the extension tube being adapted to an external injection device to pour the bone cement in liquid status and at high temperature into the extension tube at proper pressure; the taper being externally threaded on the outer circumference at one end to be meshed to the internally threaded lower end of the extension tube from the pouring frame and entering into the hole on the bone, a polygonal hole being disposed at the end of the taper connected to the extension tube, and a recession being provided at the center of the outer circumference of the taper to be adapted to a silicon gel bag containing the bone cement; and the locking rod penetrating through the extension tube of the pouring frame having at one end disposed with a polygonal protrusion to be inserted into and secured in the polygonal hole of the taper, and the lateral handle of the pouring frame being permitted to turn in reverse to disengage the externally threaded taper from the internally threaded lower end of the extension tube.

2. A vertebra bone cement introduction system of claim 1, wherein the hole on the surface of the bone relates to an inwardly flared opening , an expansion penetrating the insertion tube of the main frame; one end of the expansion rod penetrating into the hole on the bone being pivoted to a pivoting head by means of a pivot; a knob outwardly expanding from another end of the expansion rod; the expansion rod being turned by the knob; and a deflection being produced at the pivoting head to expand the interior of the hole on the bone.

3. A vertebra bone cement introduction system of claim 2, wherein a protrusion internally threaded is disposed on another side of the lateral handle of the main frame connecting the insertion tube; an externally threaded section is relatively provided on the middle section of the expansion rod; and both internal thread and external thread are meshed to push forward the expansion rod in the tube of the main frame.

4. A vertebra bone cement introduction system of claim 2, wherein the outer circumference of the knob of the expansion road is embossed.

5. A vertebra bone cement introduction system of claim 1, wherein the external injection device relates to a barrel.

6. A vertebra bone cement introduction system of claim 5, wherein the opening of the lateral handle of the pouring frame connecting through the extension is internally threaded to mesh an externally threaded end of the barrel to secure a firm incorporation of the barrel and the pouring frame.

7. A vertebra bone cement introduction system of claim 1, wherein an L-shaped handle extends from the locking rod at its end exposed from the extension tube of the pouring frame.

8. A vertebra bone cement introduction system of claim 1, wherein a T-shaped handle extends from the locking rod at its end exposed from the extension tube of the pouring frame.

9. A vertebra bone cement introduction system of claim 1, wherein multiple flanges are provided to the peripheral of the silicon gel bag.

10. A vertebra bone cement introduction system of claim 1, wherein the silicon gel bag is made in a strip form and one or a plurality of through hole is disposed on the bag.
